(19) United States
(12) Patent Application Publication
(10) Pub. No.: US 2023/0157854 A1
(43) Pub. Date: May 25, 2023

(54) STENT DELIVERY SYSTEM CAPABLE OF ACCURATELY POSITIONING

(71) Applicant: Quzhou People's Hospital, Quzhou (CN)

(72) Inventors: Xiaoyang Li, Quzhou (CN); Mengmeng Zhou, Quzhou (CN); Wei Lu, Quzhou (CN); Sheng Liao, Quzhou (CN)

(21) Appl. No.: 17/989,037

(22) Filed: Nov. 17, 2022

(30) Foreign Application Priority Data
Nov. 22, 2021 (CN) 202111385760.5

(51) Int. Cl. A61F 2/966

(52) U.S. Cl. CPC A61F 2/966

(57) ABSTRACT

The invention discloses a stent delivery system capable of accurately positioning. The system comprises a movable sheathing canal, a fixed sheathing canal and a stent, the fixed sheathing canal is used for fixing stent, the movable sheathing canal comprises a handle, a stent length buckle, an inner sheathing canal, a sheathing canal inclined part, an outer sheathing canal and an outer sheathing positioning marking part, and the sheathing canal inclined part and the outer sheathing canal are integrally formed, and are arranged at front end of inner sheathing canal. The invention provides an accurate-positioning proximal end pre-release stent delivery system and method, which is simple in structure and convenient to use. The invention can realize accurate positioning of the proximal-end of the stent, that is, the invention can prevent stent from being too long or too short due to proximal-end inaccurate release of the stent through proximal-end accurate positioning.

### TECHNICAL FIELD

The invention relates to a medical stent delivery structure, in particular to a stent delivery system capable of accurately positioning.

### BACKGROUND ART

In the invention, the term “proximal end” refers to the end close to the heart in the direction of blood flow, and “distal end” refers to the end away from the heart in the direction of blood flow. It should be understood that interventions are often accessed through the femoral artery. Bare stents are often used for lower limbs, and the stent of the invention aims to achieve precise positioning of the proximal stent, and the proximal end is released first. The covered stent is commonly used for thoracoabdominal aortic aneurysm, the purpose of the stent of the invention is to achieve precise positioning of the distal stent, and the distal end is released first.

With the rapid development of interventional therapy, it has now become the main means of treating systemic arterial diseases, and stents are an important part of interventional therapy, bare stents are often used for arterial stenosis and occlusion lesions, and covered stents are often used for arterial aneurysm and other diseases.

The current mainstream stents are mainly nickel-titanium alloy self-expanding stents, the nickel-titanium alloy stent itself has a shape memory function, the stent is loaded into the outer sheathing canal by segment-by-segment extrusion, in this process, the stent radially shrinks to the same size as the lumen of the delivery sheathing canal, however, the loaded stent is shortened in the axial direction and could not maintain the original unloaded stent length. For bare stents, if the stent is too short, it cannot completely cover the stenosis and occlusion lesions, and if the stent is too long, it may affect the bifurcated vessels and affect the surgical effect.

After the shortened stent is released, the stent may be shortened, which will lead to inaccurate distal positioning of the stent, affecting the operator's judgment, for covered stents, which may lead to excessive occlusion of the distal rupture, leading to occlusion of the bifurcated vessels close to the distal rupture, thereby affecting the blood supply of bifurcated vessels.

Therefore, a stent delivery system capable of accurately positioning has become an urgent problem to be solved in the whole society.

### SUMMARY OF THE INVENTION

To solve the above technical problems, the technical solution provided by the invention is: a stent delivery system capable of accurately positioning, comprising a movable sheathing canal, a fixed sheathing canal and a stent, wherein the fixed sheathing canal is used for fixing the stent, the movable sheathing canal comprises a handle, a stent length buckle, an inner sheathing canal, a sheathing canal inclined part, an outer sheathing canal and an outer sheathing canal positioning marking part, and the sheathing canal inclined part and the outer sheathing canal are integrally formed, and are arranged at the front end of the inner sheathing canal;

the fixed sheathing canal comprises a fixed sheathing canal side handle, a fixed sheathing middle canal, a fixed sheathing canal stent fixed buckle, a fixed nut and a contrast side hole, the fixed sheathing canal side handle is hollow inside, and is used to connect with the fixed sheathing middle canal, the fixed nut is arranged on the end of the fixed sheathing middle canal, the fixed sheathing middle canal is connected to the inner sheathing canal through the fixed nut;

a guide wire is arranged inside the inner sheathing canal.

Further, the fixed sheathing canal stent fixed buckle is arranged at outside front end of the fixed sheathing middle canal, and is used for fixing the stent, the outside of the stent is touched and connected to the outer sheathing canal, and the stent is compressed and arranged between the outer sheathing canal and the fixed sheathing middle canal.

Further, the handle is arranged at outer end of the inner sheathing canal.

Further, the stent length buckle is arranged on outer side of the inner sheathing canal, the distance from the stent length buckle to the fixed nut is the same as the length of the stent.

Further, the contrast side holes are provided at least two groups, and are arranged at the front end of the fixed sheathing canal.

Further, the fixed sheathing canal side handle is arranged on the fixed sheathing middle canal, and the bottom thereof is aslant arranged.

Further, the front end of the movable sheathing canal is connected with the front end of the sheathing canal inclined part.

Further, the handle is provided with a channel adapted to the guide wire.

Further, the length of the outer sheathing canal is adapted to the length of the stent, and the end of the outer sheathing canal is aslant arranged.

Compared to the prior art, the invention has the following technical schemes: the invention provides an accurate-positioning proximal end pre-release stent delivery system and method. The system is simple in structure and convenient to use. According to the invention, the delivery system can realize accurate positioning of the proximal end of the stent, that is, the invention can prevent the stent from being too long or too short due to inaccurate release of the proximal end of the stent through accurate positioning of the proximal end, and can prevent the stent from being too short to not completely cover a stenosis and occlusion lesions and the stent from being too long to influence bifurcated blood vessels and the surgical effect. By arranging the delivery sheath and the contrast side holes, the stent can be positioned more accurately, and the invention is worthy of being further popularized.

### BRIEF DESCRIPTION OF ACCOMPANYING DRAWINGS

FIG. 1 shows the structure of a stent delivery system capable of accurately positioning in the invention;

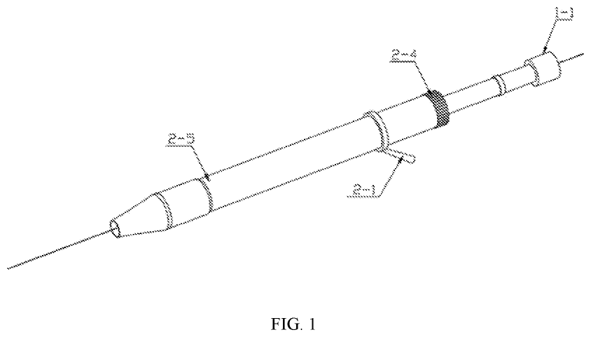

FIG. 2 shows the front view of a stent delivery system capable of accurately positioning in the invention;

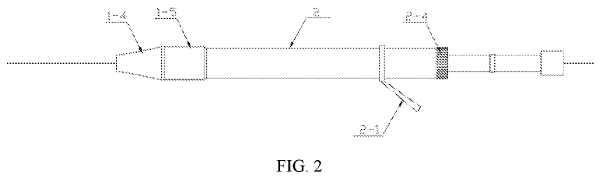

FIG. 3 shows the top view of a stent delivery system capable of accurately positioning in the invention;

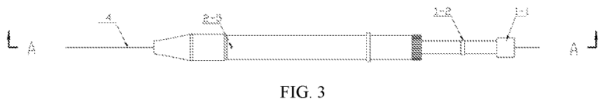

FIG. 4 shows the sectional structure at A in FIG. 3.

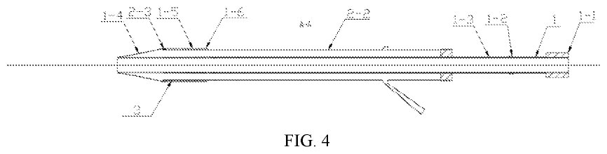

In the accompanying drawing: 1 refers to movable sheathing canal, 1-1 refers to handle. 1-2 refers to stent length buckle, 1-3 refers to inner sheathing canal, 1-4 refers to sheathing canal inclined part, 1-5 refers to outer sheathing canal, 2 refers to fixed sheathing canal, 2-1 refers to fixed sheathing canal side handle, 2-2 refers to fixed sheathing middle canal, 2-3 refers to fixed sheathing canal stent fixed buckle, 2-4 refers to fixed nut, 2-5 refers to contrast side hole, 3 refers to stent, 4 refers to guide wire.

### SPECIFIC EMBODIMENTS OF THE INVENTION

The invention is further described in detail in combination with the accompanying drawings.

Combined with the attached drawings, the invention is described in detail.

The invention provides a stent delivery system capable of accurately positioning, comprising a movable sheathing canal 1, a fixed sheathing canal 2 and a stent 3, wherein the fixed sheathing canal 2 is used for fixing the stent 3, the movable sheathing canal 1 comprises a handle 1-1, a stent length buckle 1-2, an inner sheathing canal 1-3, a sheathing canal inclined part 1-4, an outer sheathing canal 1-5 and an outer sheathing canal positioning marking part 1-6, and the sheathing canal inclined part 1-4 and the outer sheathing canal 1-5 are integrally formed, and are arranged at the front end of the inner sheathing canal 1-3;

the fixed sheathing canal 2 comprises a fixed sheathing canal side handle 2-1, a fixed sheathing middle canal 2-2, a fixed sheathing canal stent fixed buckle 2-3, a fixed nut 2-4 and a contrast side hole 2-5, the fixed sheathing canal side handle 2-1 is hollow inside, and is used to connect with the fixed sheathing middle canal 2-2, the fixed nut 2-4 is arranged on the end of the fixed sheathing middle canal 2-2, the fixed sheathing middle canal 2-2 is connected to the inner sheathing canal 1-3 through the fixed nut 2-4;

a guide wire 4 is arranged inside the inner sheathing canal 1-3.

The fixed sheathing canal stent fixed buckle 2-3 is arranged at outside front end of the fixed sheathing middle canal 2-2, and is used for fixing the stent 3, the outside of the stent 3 is touched and connected to the outer sheathing canal 1-5, and the stent 3 is compressed and arranged between the outer sheathing canal 1-5 and the fixed sheathing middle canal 2-2. The handle 1-1 is arranged at outer end of the inner sheathing canal 1-3. The stent length buckle 1-2 is arranged on outer side of the inner sheathing canal 1-3, the distance from the stent length buckle 1-2 to the fixed nut 2-4 is the same as the length of the stent 3. The contrast side holes 2-5 are provided at least two groups, and are arranged at the front end of the fixed sheathing canal 2-2. The fixed sheathing canal side handle 2-1 is arranged on the fixed sheathing middle canal 2-2, and the bottom thereof is aslant arranged. The front end of the movable sheathing canal 1 is connected with the front end of the sheathing canal inclined part 1-4. The handle 1-1 is provided with a channel adapted to the guide wire 4. The end of the outer sheathing canal 1-5 is aslant arranged.

As a further description of the invention, the length of the outer sheathing canal 1-5 is adapted to the length of the stent 3, and can just accommodate the stent 3.

As a further description of the invention, the fixed sheathing canal stent fixed buckle 2-3 is arranged at outside front end of the fixed sheathing middle canal 2-2, which is radiopaque and is convenient for positioning the head end of the stent 3.

As a further description of the invention, there is a guide wire channel in the middle of the handle 1-1, which is convenient for flushing the guide wire channel and entering the guide wire 4.

As a further description of the invention, the end of the outer sheathing canal 1-5 is aslant arranged, which can prevent the stent from being embedded with the sheathing canal when the sheathing canal is recovered after the stent 3 is released, thereby ensuring that the sheath can be withdrawn smoothly.

The specific embodiments of the invention are as follows:

Taking the occlusion of the initial segment of the superficial femoral artery as an example, the diameter of the common femoral artery is larger than that of the superficial femoral artery, in the prior art, the stent 3 pre-release system can only achieve precise distal positioning, and the positioning at the initial segment of the superficial femoral artery was poor, moreover, if the stent 3 is too long to cover the opening of the deep femoral artery, which affects the blood flow of the deep femoral artery, and if the stent 3 is too short, it is difficult to completely cover the initial lesion of the superficial femoral artery.

In the specific embodiment, when the guide wire 4 passes through the occlusion segment smoothly, the balloon dilatation preparation before the stent 3 is released has been completed, and after the common stent 3 pre-release system completes the distal positioning of the stenotic segment, the stent 3 is released, and is ready to enter the stent delivery system of the invention, before entering the vessel lumen, first flushing the guide wire 4 channel at the end of the handle with heparin water, and then tightening the fixed nut 2-4, thereby the movable sheathing canal 1 and the fixed sheathing canal 2 will not move, and avoiding releasing the stent 3 in advance, moreover, flushing the channel of the side handle of the fixed sheathing canal 2 with heparin water until the heparin water flows out of the contrast side holes 2-5.

After tightening the fixed nut 2-4, entering the stent delivery system of the invention, therefore the outer sheathing canal positioning marking part 1-6 is initially positioned at the initial segment of the superficial femoral artery, and the operator passes the contrast agent through the fixed sheathing canal side handle 2-1, the contrast agent is injected into the middle lacuna between the fixed sheathing canal 1 and the inner sheathing canal 1-3, and flows out through the contrast side holes for visualization, thereby the opening of the deep femoral artery and the superficial femoral artery can be judged again to achieve the accurate positioning of the proximal end of the stent. After repositioning, the operator loosened the tightening nuts, held the fixed sheathing canal side handle 2.1 with one hand, and moved the movable sheathing canal 1 slowly along the guide wire 4 with the other hand, the fixed sheathing canal stent fixed buckle 2-3 can help the stent to be fixed on the fixed sheathing canal 2 to avoid stent 3 from advancing with the outer sheathing canal 1-5. When movable sheathing canal 1 advances, and fixed nut 2-4 meets the stent length buckle 1-2 of movable sheathing canal 1, the stent 3 is just released completely to avoid the movable sheathing canal 1 advancing too much. At this time, the fixed sheathing canal stent fixed buckle 2-3 overlaps with the outer sheathing canal positioning marking part 1-6. After the stent 3 is released, the slope setting at the end of the outer sheathing canal 1-5 can be avoided to prevent the stent 3 from being embedded with the sheathing canal when the sheathing canal is recovered after the stent 3 is released, thereby ensuring that the sheath can be withdrawn smoothly.

The invention and embodiments thereof have been described above, and the description is not limited, and what is shown in the accompanying drawings is only one of the embodiments of the invention, and the actual structure is not limited thereto. All in all, if those of ordinary skill in the art are inspired by the embodiment, and without departing from the purpose of the invention, any structural modes and embodiments similar to the technical schemes are designed without creativity, all should belong to the protection scope of the invention.

1. A stent delivery system capable of accurately positioning, comprising a movable sheathing canal, a fixed sheathing canal and a stent, wherein the fixed sheathing canal is used for fixing the stent, the movable sheathing canal comprises a handle, a stent length buckle, an inner sheathing canal, a sheathing canal inclined part, an outer sheathing canal and an outer sheathing canal positioning marking part, and the sheathing canal inclined part and the outer sheathing canal are integrally formed, and are arranged at the front end of the inner sheathing canal;
the fixed sheathing canal comprises a fixed sheathing canal side handle, a fixed sheathing middle canal, a fixed sheathing canal stent fixed buckle, a fixed nut and contrast side holes, the fixed sheathing canal side handle is hollow inside, and is used to connect with the fixed sheathing middle canal, the fixed nut is arranged on the end of the fixed sheathing middle canal, the fixed sheathing middle canal is connected to the inner sheathing canal through the fixed nut;
a guide wire is arranged inside the inner sheathing canal.

2. The stent delivery system capable of accurately positioning of claim 1, wherein the fixed sheathing canal stent fixed buckle is arranged at outside front end of the fixed sheathing middle canal, and is used for fixing the stent, the outside of the stent is touched and connected to the outer sheathing canal, and the stent is compressed and arranged between the outer sheathing canal and the fixed sheathing middle canal.

3. The stent delivery system capable of accurately positioning of claim 1, wherein the handle is arranged at outer end of the inner sheathing canal.

4. The stent delivery system capable of accurately positioning of claim 1, wherein the stent length buckle is arranged on outer side of the inner sheathing canal, the distance from the stent length buckle to the fixed nut is the same as the length of the stent.

5. The stent delivery system capable of accurately positioning of claim 1, wherein the contrast side holes are provided at least two groups, and are arranged at the front end of the fixed sheathing canal.

6. The stent delivery system capable of accurately positioning of claim 1, wherein the fixed sheathing canal side handle is arranged on the fixed sheathing middle canal, and the bottom thereof is aslant arranged.

7. The stent delivery system capable of accurately positioning of claim 1, wherein the front end of the movable sheathing canal is connected with the front end of the sheathing canal inclined part.

8. The stent delivery system capable of accurately positioning of claim 1, the handle is provided with a channel adapted to the guide wire.

9. The stent delivery system capable of accurately positioning of claim 1, wherein the length of the outer sheathing canal is adapted to the length of the stent, and the end of the outer sheathing canal is aslant arranged.
